(19) United States
(12) Patent Application Publication
(10) Pub. No.: US 2011/0224281 A1
(43) Pub. Date: Sep. 15, 2011

(54) RNA ANTAGONISTS TARGETING HSP70-2

(76) Inventors: Anja Molhart Hog, Virum (DK); Jens Bo Hansen, Charlottenlund (DK)

(21) Appl. No.: 12/999,597

(22) PCT Filed: Jun. 18, 2009
(86) PCT No.: PCT/EP09/57569

§ 371 Date: Jun. 6, 2011

Related U.S. Application Data
(60) Provisional application No. 61/074,331, filed on Jun. 20, 2008.

(30) Foreign Application Priority Data
Jun. 19, 2008 (EP) 08104472.9

(51) Int. Cl. A61K 31/7088; C07H 21/00; A61P 35/00; C12N 5/071

(52) U.S. Cl. 514/44 A; 536/24.5; 435/375

(57) ABSTRACT

The present invention relates to LNA oligomer compounds (oligomers), which target Hsp70 and mRNA in a cell, leading to reduced expression of Hsp70. Reduction of Hsp70 expression is beneficial for the treatment of certain medical disorders, such as hyperproliferative diseases, such as cancer.

### RELATED CASES

This application claims the benefit under 35 U.S.C. §119(e) of U.S. Provisional Application Ser. No. 61/074,331 filed 20 Jun. 2008, the disclosure of which is incorporated herein by reference in its entirety.

### FIELD OF INVENTION

The present invention relates to oligomeric compounds (oligomers), which target Hsp70-2 mRNA in a cell, leading to reduced expression of Hsp70-2. Reduction of Hsp70-2 expression is beneficial for a range of medical disorders, such as hyperproliferative diseases, such as cancer.

### BACKGROUND

The human Hsp70 family contains at least eight homologous chaperone proteins that differ from each other by amino acid sequence, expression level and sub-cellular localization. The family contains housekeeping genes coding for proteins essential to homeostasis as well as stress inducible genes.

The protein products of HSPA5, HSPA8, and HSPA9 are housekeeping chaperones with functions that include transport of proteins between cellular compartments, degradation of unstable and misfolded proteins, prevention and dissolution of protein complexes, folding and refolding of proteins, uncoating of clathrincoated vesicles, and control of regulatory proteins. Confirming the essential roles of the housekeeping genes HSPA5 knockout mouse embryos die at embryonic day 3.5, while HSPA8 knockout mice mouse cannot be created due to the essential role of the protein product for cell survival. HSPA9 knockout mice have never been established but deletion of the corresponding gene in yeast is lethal.

The protein products encoded by HSPA1A and HSPA1 B, Hsp70-1a and Hsp70-1 b, (collectively referred to as Hsp70-1) are the major stress-inducible members of the family and functions as chaperones enabling the cell to cope with harmful aggregations of denatured proteins during and following stress. A long line of experimental evidence positions Hsp70-1 as a cancer relevant survival protein. It is abundantly expressed in malignant tumors of various origins and its expression correlates with increased cell proliferation, poor differentiation, lymph node metastases and poor therapeutic outcome in human breast cancer. Second, high expression of Hsp70-1 is required for the survival of tumor cells of various origins in vitro as well as for the growth of human tumour xenografts in immunodeficient mice.

The protein encoded by HSPA2, Hsp70-2, is constitutively expressed in low levels in most tissues, but in high levels in testis and brain. Male HSPA2 knockout mice are sterile due to massive germ cell apoptosis but other wise normal. Recent data indicate that Hsp70-2 is also upregulated in primary and metastatic breast cancers and has growth and survival promoting effects in cancer cells.

The antiproliferative response of a cancer cell line depleted for both Hsp70-1 and Hsp70-2 by siRNA down regulating both proteins shows much more dramatic effects than that caused by specific knock-down of either protein alone. Instead of mainly growth-arrested phenotypes observed in cells with knockdown of either Hsp70-1 and Hsp70-2 cancer cells depleted for both Hsp70-1 and Hsp70-2 undergoes massive cell death after treatment while normal cells are unaffected. A study based on a panel of small interfering RNAs (siRNAs) specifically targeting the individual family members clearly demonstrates that Hsp70-1 and Hsp70-2 have non-overlapping and specific functions related to cancer cell growth and survival while the depletion of Hsp70-1 and Hsp70-2 fails to compromise the growth of rapidly growing non-tumourigenic epithelial cells. Cancer cells depleted of Hsp70-1 or Hsp70-2 displays strikingly different morphologies (detached and round vs. flat senescent-like), cell cycle distributions (G2/M vs. G1 arrest) and gene expression profiles. There is a need for agents which can down-regulate Hsp70-2 (and optionally Hsp70-1) whilst avoiding down-regulation of other heat shock proteins, such as HSPA5, HSPA8, and HSPA9.

### SUMMARY OF INVENTION

The invention provides an oligomer of between 10-30 nucleotides in length which comprises a contiguous nucleotide sequence of a total of between 10-30 nucleotides, wherein said contiguous nucleotide sequence is at least 80% homologous to a region corresponding to one or more mammalian Hsp70 gene or mRNA, such as SEQ ID NO: 1-6 or naturally occurring variant thereof.

The invention provides an oligomer of between 10-30 nucleotides in length which comprises a contiguous nucleotide sequence of a total of between 10-30 nucleotides, wherein said contiguous nucleotide sequence is at least 80% homologous to a region corresponding to a mammalian Hsp70-2 gene or mRNA, such as SEQ ID NO: 1 or naturally occurring variant thereof.

The invention provides for a conjugate comprising the oligomer according to the invention, and at least one non-nucleotide or non-polynucleotide moiety covalently attached to said oligomer.

The invention provides for a pharmaceutical composition comprising the oligomer or the conjugate according to the invention, and a pharmaceutically acceptable diluent, carrier, salt or adjuvant.

The invention provides for the oligomer or the conjugate according to invention, for use as a medicament, such as for the treatment of hyperproliferative diseases, such as cancer.

The invention provides for the use of an oligomer or the conjugate according to the invention, for the manufacture of a medicament for the treatment of hyperproliferative diseases, such as cancer.

The invention provides for a method of treating hyperproliferative diseases, such as cancer, said method comprising administering, e.g. effective dose of, an oligomer, a conjugate or a pharmaceutical composition according to the invention, to a patient suffering from, or likely to suffer from hyperproliferative diseases, such as cancer.

The invention provides for a method for the inhibition of Hsp70-2 in a cell which is expressing Hsp70-2, said method comprising administering an oligomer, or a conjugate according to the invention to said cell so as to effect the inhibition of Hsp70-2 in said cell.

The invention provides a method for the simultaneous inhibition of Hsp70-2 and Hsp70-1 in a cell which is expressing both Hsp70-2 and Hsp70-1, said method comprising administering an oligomer, or a conjugate according to the invention to said cell so as to effect the inhibition of Hsp70-2 and Hsp70-1 in said cell.

In some aspects, the invention provides an oligomer of 10-30 monomers which consists or comprises wherein adjacent monomers are covalently linked, such as by a phosphate group or a phosphorothioate group (or another internucleotide group, such as those referred to herein), wherein said oligomer comprises a first region of at least 10 contiguous monomers; wherein at least one monomer of said first region is a nucleoside analogue; wherein the sequence of said first region is at least 80% identical to the reverse complement of the best-aligned target region of a mammalian HSP-70 gene or a mammalian HSP-70 mRNA. The sequence of the first region, may in some embodiments be identical to the sequence of a region of at least 10 contiguous monomers present in a sequence selected from SEQ ID NO 7-17 or 40-50. The sequence of the first region, may in some embodiments be identical to the sequence of a region of at least 10 contiguous monomers present in the reverse complement of a human HSP-70 gene or mRNA. In some embodiments, the first region consists of a gapmer as referred to herein.

### BRIEF DESCRIPTION OF FIGURES

FIG. 1: Alignment showing hybridisation sites of selected oligomers against the Hsp70-2 mRNA (cDNA).

FIG. 2: Oligomers presented in Table 3 were evaluated for their potential to knock down HSPA2 and HSPA1A/1B at concentrations of 1, 5 and 25 nM in A549 cells.

### DETAILED DESCRIPTION OF INVENTION

### The Oligomer

The present invention employs oligomeric compounds (referred herein as oligomers), for use in modulating the function of nucleic acid molecules encoding mammalian Hsp70-2, such as the Hsp70-2 nucleic acid shown in SEQ ID NO 1, and naturally occurring variants of such nucleic acid molecules encoding mammalian Hsp70-2.

The term “oligomer” in the context of the present invention, refers to a molecule formed by covalent linkage of two or more nucleotides (i.e. an oligonucleotide). Herein, a single nucleotide (unit) may also be referred to as a monomer or nucleic acid unit. The oligomer consists or comprises of a contiguous nucleotide sequence of between 10-30 nucleotides in length.

In various embodiments, the compound of the invention does not comprise RNA (units). It is preferred that the compound according to the invention is a linear molecule or is synthesised as a linear molecule. The oligomer is a single stranded molecule, and preferably does not comprise short regions of, for example, at least 3, 4 or 5 contiguous nucleotides, which are complementary to equivalent regions within the same oligomer (i.e. duplexes)—in this regards, the oligomer is not (essentially) double stranded. In various embodiments, the oligomer is essentially not double stranded, such as is not a siRNA. In various embodiments, the oligomer of the invention may consist entirely of the contiguous nucleotide region.

### The Target

Suitably the oligomer of the invention is capable of down-regulating expression of the Hsp70-2 gene. In this regards, the oligomer of the invention can effect the inhibition of Hsp70-2, typically in a mammalian such as a human cell. In various embodiments, the oligomers of the invention bind to the target nucleic acid and effect inhibition of expression of at least 10% or 20% compared to the normal expression level, more preferably at least a 30%, 40%, 50%, 60%, 70%, 80%, 90% or 95% inhibition compared to the normal expression level. In some embodiments, such modulation is seen when using between 0.04 and 25 nM, 1 and 25 nM, such as between 0.8 and 20 nM concentration of the compound of the invention. As illustrated herein the cell type may, in some embodiments be A549 cells (e.g. in vitro—transfected cells). The oligo concentration used (e.g. in A549) may, in some embodiments be 5 nM. The oligo concentration used may, in some embodiments be 25 nM (e.g. in A549). The oligo concentratin used may, in some embodiments be 1 nM (e.g. in A549). In the same or a different embodiment, the inhibition of expression is less than 100%, such as less than 98% inhibition, less than 95% inhibition, less than 90% inhibition, less than 80% inhibition, such as less than 70% inhibition. Modulation of expression level may be determined by measuring protein levels, e.g. by the methods such as SDS-PAGE followed by western blotting using suitable antibodies raised against the target protein. Alternatively, modulation of expression levels can be determined by measuring levels of mRNA, e.g. by northern blotting or quantitative RT-PCR. When measuring via mRNA levels, the level of down-regulation when using an appropriate dosage, such as between 0.04 and 25 nM, 1 and 25 nM, such as between 0.8 and 20 nM concentration, is, in some embodiments, typically to a level of between 10-20% the normal levels in the absence of the compound of the invention.

The invention therefore provides a method of down-regulating or inhibiting the expression of Hsp70-2 protein and/or mRNA in a cell which is expressing Hsp70-2 protein and/or mRNA, said method comprising administering the oligomer or conjugate according to the invention to said cell to down-regulating or inhibiting the expression of Hsp70-2 protein and/or mRNA in said cell. Suitably the cell is a mammalian cell such as a human cell. The administration may occur, in some embodiments, in vitro. The administration may occur, in some embodiments, in vivo. In some embodiments, the oligomer of the invention is also capable of down-regulating expression of one or more Hsp70-1 genes, such as SEQ ID NO 2 and/or 3—therefore in this embodiment the oligomer may have more than one target nucleic acid, the target may therefore be both Hsp7-2 and Hsp70-1. As illustrated in the examples, oligomers of the invention can be designed as to be essentially complementary to a corresponding region of both Hsp70-1 and Hsp70-2, although this may require the use of one or two mismatches to the corresponding Hsp70-1 or Hsp70-2 sequence (i.e. essentially complementary).

In some embodiments, the oligomer of the invention is not capable of down-regulating Hsp70-5, Hsp70-8 or Hsp70-9, such as SEQ ID NO 4, 5 or 6—these nucleic acids are, therefore, in this embodiment, not the target nucleic acid.

The term “target nucleic acid”, as used herein refers to DNA or RNA encoding mammalian Hsp70-2 polypeptide, such as human Hsp70-2, such as SEQ ID NO 1. It will be recognised that in respect to oligomers which also target Hsp70-1, the target nucleic acid may also be DNA or RNA encoding mammalian Hsp70-1 polypeptide(s), such as human Hsp70-1A and/or Hsp70-1 B, such as SEQ ID NO 2 and/or 3.

It will be recognised that SEQ ID NOs 1-6 are cDNA sequences, and as such, correspond to the mature mRNA target sequence, although uracil is replaced with thymidine in the cDNA sequences. The target nucleic acid is preferably the RNA nucleic acid corresponding to the cDNA sequence—i.e. preferably mRNA, such as pre-mRNA, although preferably mature mRNA. In some embodiments, for example when used in research or diagnostics the “target nucleic acid” may be a cDNA or a synthetic oligonucleotide derived from the above DNA or RNA nucleic acid targets. The oligomer according to the invention is preferably capable of hybridising to the target nucleic acid.

The term “naturally occurring variant thereof” refers to variants of the Hsp70 (such as HSP-70-2) polypeptide of nucleic acid sequence which exist naturally within the defined taxonomic group, such as mammalian, such as mouse, monkey, and preferably human. Typically, when referring to “naturally occurring variants” of a polynucleotide the term also may encompass any allelic variant of the human Hsp70-2 encoding genomic DNA which is found at the human Chromosome: 14; Location: 14q24.1 Mb, and the RNA, such as mRNA derived therefrom. “Naturally occurring variants” may also include variants derived from alternative splicing of the Hsp70-2 mRNA. For oligonucleotides which also target the Hsp-70-1 mRNA, an allelic variant of the human Hsp70-1 encoding genomic DNA is found at the human Chromosome: 6; Location: Mb 6p21.3. When referenced to a specific polypeptide sequence, e.g., the term also includes naturally occurring forms of the protein which may therefore be processed, e.g. by co- or post-translational modifications, such as signal peptide cleavage, proteolytic cleavage, glycosylation, etc.

### Sequences

The oligomers comprise or consist of a contiguous nucleotide sequence which corresponds to a nucleotide sequence present in SEQ ID NO: 1, or a sequence selected from the group consisting of SEQ ID NOS: 7-28 and 40-50, wherein said oligomer (or contiguous nucleotide portion thereof) may optionally comprise one, two, or three mismatches against said selected sequence.

The oligomer may comprise or consist of a contiguous nucleotide sequence which is fully complementary (perfectly complementary) to the equivalent region of a nucleic acid which encodes a mammalian Hsp70-2—i.e. comprises an antisense nucleotide sequence.

However, in some embodiments, the oligomer may tolerate 1, 2, 3, or 4 (or more) mismatches, when hybridising to the target sequence and still sufficiently bind to the target to show the desired effect, i.e. down-regulation of the target. Mismatches may, for example, be compensated by increased length of the oligomer nucleotide sequence and/or an increased number of nucleotide analogues, such as LNA, present within the nucleotide sequence.

In some embodiments, the contiguous nucleotide sequence comprises no more than 3, such as no more than 2 mismatches to the target sequence, such as to the corresponding region of a nucleic acid which encodes a mammalian Hsp70-2.

In some embodiments, the contiguous nucleotide sequence comprises no more than a single mismatch to the target sequence, such as the corresponding region of a nucleic acid which encodes a mammalian Hsp70-2.

The nucleotide sequence of the oligomers of the invention or the contiguous nucleotide sequence is preferably at least 80% homologous to a corresponding sequence selected from the group consisting of SEQ ID NOS: 7-28 and 40-50, such as at least 85%, at least 90%, at least 91%, at least 92%, at least 93%, at least 94%, at least 95%, at least 96% homologous, such as 100% homologous (identical).

The nucleotide sequence of the oligomers of the invention or the contiguous nucleotide sequence is preferably at least 80% homologous to a corresponding sequence present in SEQ ID NO: 1, such as at least 85%, at least 90%, at least 91%, at least 92% at least 93%, at least 94%, at least 95%, at least 96% homologous, such as 100% homologous (identical).

The nucleotide sequence of the oligomers of the invention or the contiguous nucleotide sequence is preferably at least 80% complementary to a sub-sequence present in SEQ ID NO: 1, such as at least 85%, at least 90%, at least 91%, at least 92% at least 93%, at least 94%, at least 95%, at least 96% complementary, such as 100% complementary (perfectly complementary).

In various embodiments the oligomer (or contiguous nucleotide portion thereof) is selected from, or comprises, one of the sequences selected from the group consisting of SEQ ID NOS: 7, 8, 9, 10, 11, 12, 13, 14, 15, 16, 17 or a sub-sequence of at least 10 contiguous nucleotides thereof, wherein said oligomer (or contiguous nucleotide portion thereof) may optionally comprise one, two, or three mismatches against said selected sequence.

In various embodiments the oligomer (or contiguous nucleotide portion thereof) is selected from, or comprises, one of the sequences selected from the group consisting of SEQ ID NOS: 51, 52, 53, 54, 55, 56, 57, 58, 59, 61, 61 or a sub-sequence of at least 10 contiguous nucleotides thereof, wherein said oligomer (or contiguous nucleotide portion thereof) may optionally comprise one, two, or three mismatches against said selected sequence.

In various embodiments the sub-sequence may consist of 11, 12, 13, 14, 15, 16, 17, 18, 19, 20, 21, 22, 23, or 24, contiguous nucleotides, such as between 12-22, such as between 12-18 nucleotides. Suitably, in various embodiments, the sub-sequence is of the same length as the contiguous nucleotide sequence of the oligomer of the invention.

However, it is recognised that, in various embodiments the nucleotide sequence of the oligomer may comprise additional 5′ or 3′ nucleotides, such as, independently, 1, 2, 3, 4 or 5 additional nucleotides 5′ and/or 3′, which are non-complementary to the target sequence. In this respect the oligomer of the invention, may, in various embodiments, comprise a contiguous nucleotide sequence which is flanked 5′ and or 3′ by additional nucleotides. In various embodiments the additional 5′ or 3′ nucleotides are naturally occurring nucleotides, such as DNA or RNA. In various embodiments, the additional 5′ or 3′ nucleotides may represent region D as referred to in the context of gapmer oligomers herein.

In various embodiments the oligomer according to the invention consists or comprises of a nucleotide sequence according to SEQ ID NO:7, or a sub-sequence of thereof.

In various embodiments the oligomer according to the invention consists or comprises of a nucleotide sequence according to SEQ ID NO:8, or a sub-sequence of thereof.

In various embodiments the oligomer according to the invention consists or comprises of a nucleotide sequence according to SEQ ID NO:9, or a sub-sequence of thereof.

In various embodiments the oligomer according to the invention consists or comprises of a nucleotide sequence according to SEQ ID NO: 10, or a sub-sequence of thereof.

In various embodiments the oligomer according to the invention consists or comprises of a nucleotide sequence according to SEQ ID NO:11, or a sub-sequence of thereof.

In various embodiments the oligomer according to the invention consists or comprises of a nucleotide sequence according to SEQ ID NO:12, or a sub-sequence of thereof.

In various embodiments the oligomer according to the invention consists or comprises of a nucleotide sequence according to SEQ ID NO:13, or a sub-sequence of thereof.

In various embodiments the oligomer according to the invention consists or comprises of a nucleotide sequence according to SEQ ID NO:14, or a sub-sequence of thereof.

In various embodiments the oligomer according to the invention consists or comprises of a nucleotide sequence according to SEQ ID NO:15, or a sub-sequence of thereof.

In various embodiments the oligomer according to the invention consists or comprises of a nucleotide sequence according to SEQ ID NO:16, or a sub-sequence of thereof.

In various embodiments the oligomer according to the invention consists or comprises of a nucleotide sequence according to SEQ ID NO:17, or a sub-sequence of thereof.

In various embodiments the oligomer according to the invention consists or comprises of a nucleotide sequence according to SEQ ID NO:18, or a sub-sequence of thereof.

When determining “homology” or “complementarity” between the oligomers of the invention (or contiguous nucleotide sequence) and the nucleic acid which encodes the mammalian Hsp70-2 (or target nucleic acid), such as those disclosed herein, the determination of homology may be made by a simple alignment with the corresponding nucleotide sequence of the compound of the invention and the corresponding region of the nucleic acid which encodes the mammalian Hsp70-2 (or target nucleic acid), or complement thereof, and the homology is determined by counting the number of bases which align and dividing by the total number of contiguous nucleotides in the compound of the invention, and multiplying by 100. In such a comparison, if gaps exist, it is preferable that such gaps are merely mismatches rather than areas where the number of nucleotides within the gap differ between the nucleotide sequence of the invention and the target nucleic acid.

The terms “corresponding to” and “corresponds to” refer to the comparison between the nucleotide sequence of the oligomer or contiguous nucleotide sequence (a first sequence) and the equivalent contiguous nucleotide sequence of a further sequence selected from either i) a sub-sequence of the reverse complement of the nucleic acid target, such as the mRNA which encodes the Hsp70-2 protein, such as SEQ ID NO: 1, and/or ii) the sequence of nucleotides provided herein such as the group consisting of SEQ ID NOS: 7-28 and 40-50. Nucleotide analogues are compared directly to their equivalent or corresponding nucleotides. A first sequence which corresponds to a further sequence under i) or ii) typically is identicial to that sequence over the length of the first sequence (such as the contiguous nucleotide sequence) or, as described herein may, in some embodiments, is at least 80% homologous to a corresponding sequence, such as at least 85%, at least 90%, at least 91%, at least 92% at least 93%, at least 94%, at least 95%, at least 96% homologous, such as 100% homologous (identical). The terms “corresponding nucleotide analogue” and “corresponding nucleotide” are intended to indicate that the nucleotide in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine.

### Length

The oligomers comprise or consist of a contiguous nucleotide sequence of a total of between 10, 11, 12, 13, 14, 15, 16, 17, 18, 19, 20, 21, 22, 23, 24, 25, 26, 27, 28, 29 or 30 contiguous nucleotides in length.

In various embodiments, the oligomers comprise or consist of a contiguous nucleotide sequence of a total of between 10-22, such as 12-18, such as 13-17 or 12-16, such as 13, 14, 15, 16 contiguous nucleotides in length.

In various embodiments, the oligomers comprise or consist of a contiguous nucleotide sequence of a total of 10, 11, 12, 13, or 14 contiguous nucleotides in length.

In various embodiments, the oligomer according to the invention consists of no more than 22 nucleotides, such as no more than 20 nucleotides, such as no more than 18 nucleotides, such as 15, 16 or 17 nucleotides. In various embodiments the oligomer of the invention comprises less than 20 nucleotides.

### Nucleotide Analogues

The term “nucleotide” as used herein, refers to a glycoside comprising a sugar moiety, a base moiety and a covalently linked group, such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogues” herein. Herein, a single nucleotide (unit) may also be referred to as a monomer or nucleic acid unit.

In field of biochemistry, the term “nucleoside” is commonly used to refer to a glycoside comprising a sugar moiety and a base moiety, and may therefore be used when referring to the nucleotide units, which are covalently linked by the internucleotide linkages between the nucleotides of the oligomer.

As one of ordinary skill in the art would recognise, the 5′ nucleotide of an oligonucleotide does not comprise a 5′ internucleotide linkage group, although may or may not comprise a 5′ terminal group.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier & Altmann; Nucli. Acid Res., 1997, 25, 4429-4443 and Uhlmann; Curr. Opinion in Drug Development, 2000, 3(2), 293-213, and in Scheme 1 (below):

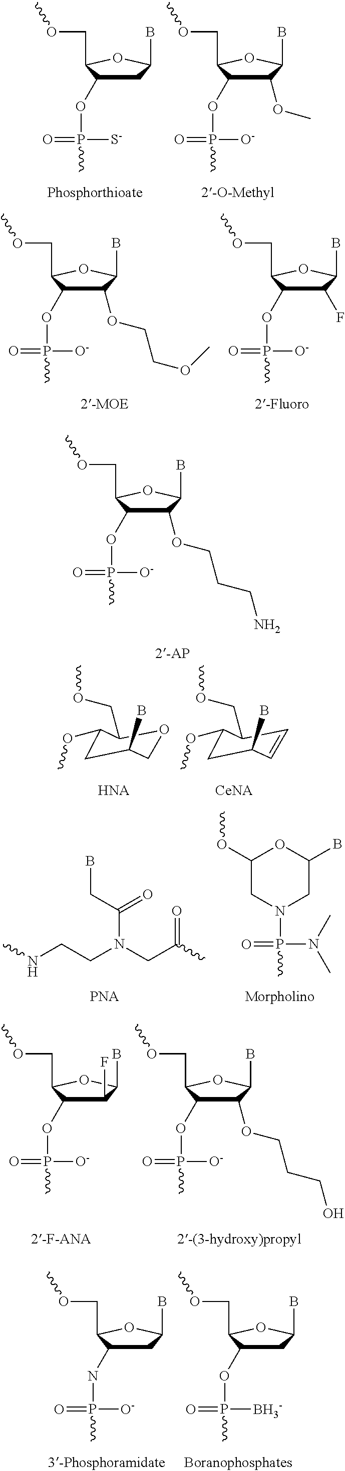

The oligomer may thus comprise or consist of a simple sequence of natural occurring nucleotides—preferably 2′-deoxynucleotides (referred to here generally as “DNA”), but also possibly ribonucleotides (referred to here generally as “RNA”), or a combination of such naturally occurring nucleotides and one or more non-naturally occurring nucleotides, i.e. nucleotide analogues. Such nucleotide analogues may suitably enhance the affinity of the oligomer for the target sequence.

Examples of suitable and preferred nucleotide analogues are provided by PCT/DK2006/000512 or are referenced therein.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

In various embodiments the oligomer comprises at least 2 nucleotide analogues. In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA.

It will be recognised that when referring to a preferred nucleotide sequence motif or nucleotide sequence, which consists of only nucleotides, the oligomers of the invention which are defined by that sequence may comprise a corresponding nucleotide analogue in place of one or more of the nucleotides present in said sequence, such as LNA units or other nucleotide analogues, which raise the duplex stability/Tₘ of the oligomer/target duplex (i.e. affinity enhancing nucleotide analogues).

In various embodiments, any mismatches between the nucleotide sequence of the oligomer and the target sequence are preferably found in regions outside the affinity enhancing nucleotide analogues, such as region B as referred to herein, and/or region D as referred to herein, and/or at the site of non modified such as DNA nucleotides in the oligonucleotide, and/or in regions which are 5′ or 3′ to the contiguous nucleotide sequence.

Examples of such modification of the nucleotide include modifying the sugar moiety to provide a 2′-substituent group or to produce a bridged (locked nucleic acid) structure which enhances binding affinity and may also provide increased nuclease resistance.

A preferred nucleotide analogue is LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). Most preferred is beta-D-oxy-LNA.

In some embodiments the nucleotide analogues present within the oligomer of the invention (such as in regions A and C mentioned herein) are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid—Christensen, 2002. Nucl. Acids. Res. 2002 30: 4918-4925, hereby incorporated by reference) units and 2′MOE units. In various embodiments there is only one of the above types of nucleotide analogues present in the oligomer of the invention, or contiguous nucleotide sequence thereof.

In various embodiments the nucleotide analogues are 2′-O-methoxyethyl-RNA (2′MOE), 2′-fluoro-DNA monomers or LNA nucleotide analogues, and as such the oligonucleotide of the invention may comprise nucleotide analogues which are independently selected from these three types of analogue, or may comprise only one type of analogue selected from the three types. In various embodiments at least one of said nucleotide analogues is 2′-MOE-RNA, such as 2, 3, 4, 5, 6, 7, 8, 9 or 10 2′-MOE-RNA nucleotide units. In various embodiments at least one of said nucleotide analogues is 2′-fluoro DNA, such as 2, 3, 4, 5, 6, 7, 8, 9 or 10 2′-fluoro-DNA nucleotide units.

In various embodiments, the oligomer according to the invention comprises at least one Locked Nucleic Acid (LNA) unit, such as 1, 2, 3, 4, 5, 6, 7, or 8 LNA units, such as between 3-7 or 4 to 8 LNA units, or 3, 4, 5, 6 or 7 LNA units. In various embodiments, all the nucleotide analogues are LNA. In some embodiments, the oligomer may comprise both beta-D-oxy-LNA, and one or more of the following LNA units: thio-LNA, amino-LNA, oxy-LNA, and/or ENA in either the beta-D or alpha-L configurations or combinations thereof. In various embodiments all LNA cytosine units are 5′ methyl-Cytosine. In various embodiments of the invention, the oligomer may comprise both LNA and DNA units. Preferably the combined total of LNA and DNA units is 10-25, preferably 10-20, even more preferably 12-16. In various embodiments of the invention, the nucleotide sequence of the oligomer, such as the contiguous nucleotide sequence consists of at least one LNA and the remaining nucleotide units are DNA units. In various embodiments the oligomer comprises only LNA nucleotide analogues and naturally occurring nucleotides (such as RNA or DNA, most preferably DNA nucleotides), optionally with modified internucleotide linkages such as phosphorothioate.

The term “nucleobase” refers to the base moiety of a nucleotide and covers both naturally occurring a well as non-naturally occurring variants. Thus, “nucleobase” covers not only the known purine and pyrimidine heterocycles but also heterocyclic analogues and tautomeres thereof.

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

In various embodiments, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

### LNA

The term “LNA” refers to a bicyclic nucleotide analogue, known as “Locked Nucleic Acid”. It may refer to an LNA monomer, or, when used in the context of an “LNA oligonucleotide”, LNA refers to an oligonucleotide containing one or more such bicyclic nucleotide analogues. LNA nucleotides are characterised by the presence of a biradical ‘bridge’ between C2′ and C4′ of the ribose sugar ring—for example as shown as the biradical R⁴*— R²* as described below.

The LNA used in the oligonucleotide compounds of the invention preferably has the structure of the general formula I

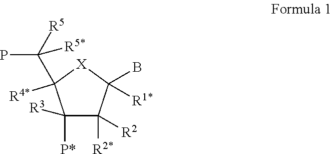

wherein for all chiral centers, asymmetric groups may be found in either R or S orientation;

wherein X is selected from —O—, —S—, —N(R^{N}*)—, —C(R⁶R⁶*)—, such as, in some embodiments —O—;

B is selected from hydrogen, optionally substituted C₁₋₄-alkoxy, optionally substituted C₁₋₄-alkyl, optionally substituted C₁₋₄-acyloxy, nucleobases including naturally occurring and nucleobase analogues, DNA intercalators, photochemically active groups, thermochemically active groups, chelating groups, reporter groups, and ligands;

P designates an internucleotide linkage to an adjacent monomer, or a 5′-terminal group, such internucleotide linkage or 5′-terminal group optionally including the substituent R⁵ or equally applicable the substituent R⁵*;

P* designates an internucleotide linkage to an adjacent monomer, or a 3′-terminal group;

R⁴* and R²* together designate a biradical consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z, wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, optionally substituted C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), wherein for all chiral centers, asymmetric groups may be found in either R or S orientation, and;

each of the substituents R¹*, R², R³, R⁵, R⁵*, R⁶ and R⁶*, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene; wherein R^{N} is selected from hydrogen and C₁₋₄-alkyl, and where two adjacent (non-geminal) substituents may designate an additional bond resulting in a double bond; and R^{N*}, when present and not involved in a biradical, is selected from hydrogen and C₁₋₄-alkyl; and basic salts and acid addition salts thereof. For all chiral centers, asymmetric groups may be found in either R or S orientation.

In some embodiments, R⁴* and R²* together designate a biradical consisting of a groups selected from the group consisting of C(R^{a}R^{b})—C(R^{a}R^{b})—, C(R^{a}R^{b})—O—, C(R^{a}R^{b})—NR^{a}—, C(R^{a}R^{b})—S—, and C(R^{a}R^{b})—C(R^{a}R^{b})—O—, wherein each R^{a} and R^{b} may optionally be independently selected. In some embodiments, R^{a} and R^{b} may be, optionally independently selected from the group consisting of hydrogen and C₁₋₆alkyl, such as methyl, such as hydrogen.

In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆alkynyl or substituted C₂₋₆alkynyl, C₁₋₆ alkoxyl, substituted C₁₋₆alkoxyl, acyl, substituted acyl, C₁₋₆-aminoalkyl or substituted C₁₋₆-aminoalkyl. For all chiral centers, asymmetric groups may be found in either R or S orientation.

In some embodiments, R¹*, R², R³, R⁵, R⁵* are hydrogen.

In some embodiments, R¹*, R², R³ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆alkynyl or substituted C₂₋₆alkynyl, C₁₋₆ alkoxyl, substituted C₁₋₆ alkoxyl, acyl, substituted acyl, C₁₋₆-aminoalkyl or substituted C₁₋₆-aminoalkyl. For all chiral centers, asymmetric groups may be found in either R or S orientation.

In some embodiments, R¹*, R², R³ are hydrogen.

In some embodiments, R⁵ and R⁵* are each independently selected from the group consisting of H, —CH₃, —CH₂—CH₃, —CH₂—O—CH₃, and —CH═CH₂. Suitably in some embodiments, either R⁵ or R⁵* are hydrogen, where as the other group (R⁵ or R^{5*} respectively) is selected from the group consisting of C₁₋₅ alkyl, C₂₋₆ alkenyl, C₂₋₆alkynyl, substituted C₁₋₆ alkyl, substituted C₂₋₆ alkenyl, substituted C₂₋₆alkynyl or substituted acyl (—C(═O)—); wherein each substituted group is mono or poly substituted with substituent groups independently selected from halogen, C₁₋₆ alkyl, substituted C₁₋₆ alkyl, C₂₋₆ alkenyl, substituted C₂₋₆ alkenyl, C₂₋₆alkynyl, substituted C₂₋₆alkynyl, OJ₁, SJ₁, NJ₁J₂, N₃, COOJ₁, CN, O—C(═O)NJ₁J₂, N(H)C(═NH)NR, R₂ or N(H)C(═X)N(H)J₂ wherein X is O or S; and each J₁ and J₂ is, independently, H, C₁₋₆ alkyl, substituted C₁₋₆ alkyl, C₂₋₆ alkenyl, substituted C₂₋₆ alkenyl, C₂₋₆alkynyl, substituted C₂₋₆ alkynyl, C₁₋₆ aminoalkyl, substituted C₁₋₆ aminoalkyl or a protecting group. In some embodiments either R⁵ or R⁵* is substituted C₁₋₆ alkyl. In some embodiments either R⁵ or R⁵* is substituted methylene wherein preferred substituent groups include one or more groups independently selected from F, NJ₁J₂, N₃, CN, OJ₁, SJ₁, O—C(═O)NJ₁J₂, N(H)C(═NH)NJ, J₂ or N(H)C(O)N(H)J₂. In some embodiments each J₁ and J₂ is, independently H or C₁₋₆ alkyl. In some embodiments either R⁵ or R⁵* is methyl, ethyl or methoxymethyl. In some embodiments either R⁵ or R⁵* is methyl. In a further embodiment either R⁵ or R⁵* is ethylenyl. In some embodiments either R⁵ or R⁵* is substituted acyl. In some embodiments either R⁵ or R⁵* is C(═O)NJ₁J₂. For all chiral centers, asymmetric groups may be found in either R or S orientation. Such 5′ modified bicyclic nucleotides are disclosed in WO 2007/134181, which is hereby incorporated by reference in its entirety.

In some embodiments B is a nucleobase, including nucleobase analogues and naturally occurring nucleobases, such as a purine or pyrimidine, or a substituted purine or substituted pyrimidine, such as a nucleobase referred to herein, such as a nucleobase selected from the group consisting of adenine, cytosine, thymine, adenine, uracil, and/or a modified or substituted nucleobase, such as 5-thiazolo-uracil, 2-thio-uracil, 5-propynyl-uracil, 2′ thio-thymine, 5-methyl cytosine, 5-thiozolo-cytosine, 5-propynyl-cytosine, and 2,6-diaminopurine.

In some embodiments, R⁴* and R²* together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{e}R^{b})—C(R^{c}R^{d})—N(R^{e})—, —C(R^{e}R^{b})—N(R^{c})—O—, and —C(R^{a}R^{b})—S—, —C(R^{a}R^{b})—C(R^{c}R^{d})—S—, wherein R^{a}, R^{b}, R^{c}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂). For all chiral centers, asymmetric groups may be found in either R or S orientation.

In a further embodiment R⁴* and R²* together designate a biradical (bivalent group) selected from —CH₂—O—, —CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, and —CH(CH₂—O—CH₃)—O—, and/or, —CH₂—CH₂—, and —CH═CH—For all chiral centers, asymmetric groups may be found in either R or S orientation.

In some embodiments, R⁴* and R²* together designate the biradical C(R^{a}R^{b})—N(R^{c})—O—, wherein R^{a} and R^{b} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen, and; wherein R^{c} is selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* together designate the biradical C(R^{a}R^{b})—O—C(R^{c}R^{d}) —O—, wherein R^{a}, R^{b}, R^{c}, and R^{d} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* form the biradical —CH(Z)—O—, wherein Z is selected from the group consisting of C₁₋₆ alkyl, C₂₋₆ alkenyl, C₂₋₆ alkynyl, substituted C₁₋₆ alkyl, substituted C₂₋₆ alkenyl, substituted C₂₋₆ alkynyl, acyl, substituted acyl, substituted amide, thiol or substituted thio; and wherein each of the substituted groups, is, independently, mono or poly substituted with optionally protected substituent groups independently selected from halogen, oxo, hydroxyl, OJ₁, NJ₁J₂, SJ₁, N₃, OC(═X)J₁, OC(═X)NJ₁J₂, NJ³C(═X)NJ₁J₂ and CN, wherein each J₁, J₂ and J₃ is, independently, H or C₁₋₆ alkyl, and X is O, S or NJ₁. In some embodiments Z is C₁₋₆ alkyl or substituted C₁₋₆ alkyl. In some embodiments Z is methyl.

In some embodiments Z is substituted C₁₋₆ alkyl. In some embodiments said substituent group is C₁₋₆alkoxy. In some embodiments Z is CH₃OCH₂—. For all chiral centers, asymmetric groups may be found in either R or S orientation. Such bicyclic nucleotides are disclosed in U.S. Pat. No. 7,399,845 which is hereby incorporated by reference in its entirety. In some embodiments, R¹*, R², R³, R⁵, R⁵* are hydrogen. In some some embodiments, R¹*, R², R³* are hydrogen, and one or both of R⁵, R⁵* may be other than hydrogen as referred to above and in WO 2007/134181.

In some embodiments, R⁴* and R²* together designate a biradical which comprise a substituted amino group in the bridge such as consist or comprise of the biradical —CH₂—N(R^{c})—, wherein R^{c} is C₁₋₁₂ alkyloxy. In some embodiments R⁴* and R²* together designate a biradical -Cq₃q₄—NOR—, wherein q₃ and q₄ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆-aminoalkyl or substituted C₁₋₆ aminoalkyl; wherein each substituted group is, independently, mono or poly substituted with substituent groups independently selected from halogen, OJ₁, SJ₁, NJ₁J₂, COOJ₁, CN, O—C(═O)NJ₁J₂, N(H)C(═NH)N J₁J₂ or N(H)C(═X═N(H)J₂ wherein X is O or S; and each of J₁ and J₂ is, independently, H, C₁₋₆ alkyl, C₂₋₆ alkenyl, C₂₋₆ alkynyl, C₁₋₆ aminoalkyl or a protecting group. For all chiral centers, asymmetric groups may be found in either R or S orientation. Such bicyclic nucleotides are disclosed in WO2008/150729 which is hereby incorporated by reference in its entirity. In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆alkoxyl, substituted C₁₋₆ alkoxyl, acyl, substituted acyl, C₁₋₆-aminoalkyl or substituted C₁₋₆ aminoalkyl. In some embodiments, R¹*, R², R³, R⁵, R⁵* are hydrogen. In some embodiments, R¹*, R², R³ are hydrogen and one or both of R⁵, R⁵* may be other than hydrogen as referred to above and in WO 2007/134181. In some embodiments R⁴* and R²* together designate a biradical (bivalent group) C(R^{a}R^{b})—O—, wherein R^{a} and R^{b} are each independently halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ₁ SJ₁, SOJ₁, SO₂J₁, NJ₁J₂, N₃, CN, C(═O)OJ₁, C(═O)NJ₁J₂, C(═O)J₁, O—C(═O)NJ₁J₂, N(H)C(═NH)NJ₁J₂, N(H)C(═O)NJ₁J₂ or N(H)C(═S)NJ₁J₂; or R^{a} and R^{b} together are ═C(q3)(q4); q₃ and q₄ are each, independently, H, halogen, C₁-C₁₂alkyl or substituted C₁-C₁₂ alkyl; each substituted group is, independently, mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ₁J₂, N₃, CN, C(═O)OJ₁, C(═O)NJ₁J₂, C(═O)J₁, O—C(═O)NJ₁J₂, N(H)C(═O)NJ₁J₂ or N(H)C(═S)NJ₁J₂ and; each J₁ and J₂ is, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₁-C₆ aminoalkyl, substituted C₁-C₆ aminoalkyl or a protecting group. Such compounds are disclosed in WO2009006478A, hereby incorporated in its entirety by reference.

In some embodiments, R⁴* and R²* form the biradical -Q-, wherein Q is C(q1)(q2)C(q3)(q4), C(q₁)=C(q₃), C[═C(q₁)(q₂)]-C(q₃)(q₄) or C(q₁)(q₂)-C[═C(q₃)(q₄)]; q₁, q₂, q₃, q₄ are each independently. H, halogen, C₁₋₁₂ alkyl, substituted C₁₋₁₂ alkyl, C₂₋₁₂ alkenyl, substituted C₁₋₁₂ alkoxy, OJ₁, SJ₁, SOJ₁, SO₂J₁, NJ₁J₂, N₃, CN, C(═O)OJ₁, C(═O)—NJ₁J₂, C(═O) J₁, —C(═O)NJ₁J₂, N(H)C(═NH)NJ₁J₂, N(H)C(═O)NJ₁J₂ or N(H)C(═S)NJ₁J₂; each J₁ and J₂ is, independently, H, C₁₋₆ alkyl, C₂₋₆ alkenyl, C₂₋₆ alkynyl, C₁₋₆ aminoalkyl or a protecting group; and, optionally wherein when Q is C(q₁)(q₂)(q₃)(q₄) and one of q₃ or q₄ is CH₃ then at least one of the other of q₃ or q₄ or one of q₁ and q₂ is other than H. In some embodiments, R¹*, R², R³, R⁵, R⁵* are hydrogen. For all chiral centers, asymmetric groups may be found in either R or S orientation. Such bicyclic nucleotides are disclosed in WO2008/154401 which is hereby incorporated by reference in its entirity. In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆alkoxyl, substituted C₁₋₆alkoxyl, acyl, substituted acyl, C₁₋₆-aminoalkyl or substituted C₁₋₆ aminoalkyl. In some embodiments, R¹*, R², R³, R⁵, R⁵* are hydrogen. In some embodiments, R¹*, R², R³ are hydrogen and one or both of R⁵, R⁵* may be other than hydrogen as referred to above and in WO 2007/134181 or WO2009/067647 (alpha-L-bicyclic nucleic acids analogs).

In some embodiments the LNA used in the oligonucleotide compounds of the invention preferably has the structure of the general formula II:

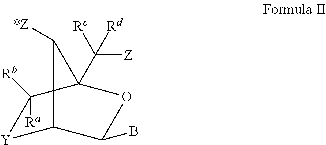

wherein Y is selected from the group consisting of —O—, —CH₂O—, —S—, —NH—, N(R^{e}) and/or —CH₂—; Z and Z* are independently selected among an internucleotide linkage, R^{H}, a terminal group or a protecting group; B constitutes a natural or non-natural nucleotide base moiety (nucleobase), and R^{H} is selected from hydrogen and C₁₋₄-alkyl; R^{a}, R^{b} R^{c}, R^{d} and R^{e} are, optionally independently, selected from the group consisting of hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂); and R^{H} is selected from hydrogen and C₁₋₄-alkyl. In some embodiments R^{a}, R^{b} R^{c}, R^{d} and R^{e} are, option ally independently, selected from the group consisting of hydrogen and C₁₋₆ alkyl, such as methyl. For all chiral centers, asymmetric groups may be found in either R or S orientation, for example, two exemplary stereochemical isomers include the beta-D and alpha-L isoforms, which may be illustrated as follows:

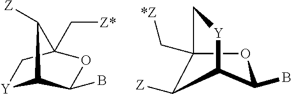

Specific exemplary LNA units are shown below:

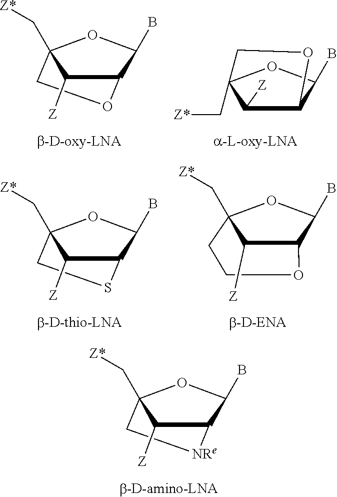

The term “thio-LNA” comprises a locked nucleotide in which Y in the general formula above is selected from S or —CH₂—S—. Thio-LNA can be in both beta-D and alpha-L-configuration.

The term “amino-LNA” comprises a locked nucleotide in which Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which Y in the general formula above represents —O—. Oxy-LNA can be in both beta-D and alpha-L-configuration.

The term “ENA” comprises a locked nucleotide in which Y in the general formula above is —CH₂—O— (where the oxygen atom of —CH₂—O— is attached to the 2′-position relative to the base B). R^{e} is hydrogen or methyl.

In some exemplary embodiments LNA is selected from beta-D-oxy-LNA, alpha-L-oxy-LNA, beta-D-amino-LNA and beta-D-thio-LNA, in particular beta-D-oxy-LNA.

RNAse Recruitment

It is recognised that an oligomeric compound may function via non RNase mediated degradation of target mRNA, such as by steric hindrance of translation, or other methods, however, the preferred oligomers of the invention are capable of recruiting an endoribonuclease (RNase), such as RNase H.

It is preferable that the oligomer, or contiguous nucleotide sequence, comprises of a region of at least 6, such as at least 7 consecutive nucleotide units, such as at least 8 or at least 9 consecutive nucleotide units (residues), including 7, 8, 9, 10, 11, 12, 13, 14, 15 or 16 consecutive nucleotides, which, when formed in a duplex with the complementary target RNA is capable of recruiting RNase. The contiguous sequence which is capable of recruiting RNAse may be region B as referred to in the context of a gapmer as described herein. In various embodiments the size of the contiguous sequence which is capable of recruiting RNAse, such as region B, may be higher, such as 10, 11, 12, 13, 14, 15, 16, 17, 18, 19 or 20 nucleotide units.

EP 1 222 309 provides in vitro methods for determining RNaseH activity, which may be used to determine the ability to recruit RNaseH. A oligomer is deemed capable of recruiting RNase H if, when provided with the complementary RNA target, it has an initial rate, as measured in pmol/l/min, of at least 1%, such as at least 5%, such as at least 10% or less than 20% of the equivalent DNA only oligonucleotide, with no 2′ substitutions, with phosphorothioate linkage groups between all nucleotides in the oligonucleotide, using the methodology provided by Example 91-95 of EP 1 222 309.

In various embodiments, an oligomer is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

In other embodiments, an oligomer is deemed capable of recruiting RNaseH if, when provided with the complementary RNA target, and RNaseH, the RNaseH initial rate, as measured in pmol/l/min, is at least 20%, such as at least 40%, such as at least 60%, such as at least 80% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

Typically the region of the oligomer which forms the consecutive nucleotide units which, when formed in a duplex with the complementary target RNA is capable of recruiting RNase consists of nucleotide units which form a DNA/RNA like duplex with the RNA target—and include both DNA units and LNA units which are in the alpha-L configuration, particularly preferred being alpha-L-oxy LNA.

The oligomer of the invention may comprise a nucleotide sequence which comprises both nucleotides and nucleotide analogues, and may be in the form of a gapmer, a headmer or a mixmer.

A headmer is defined by a contiguous stretch of non-RNase recruiting nucleotide analogues at the 5′-end followed by a contiguous stretch of DNA or modified nucleotide units recognizable and cleavable by the RNase towards the 3′-end (such as at least 7 such nucleotides), and a tailmer is defined by a contiguous stretch of DNA or modified nucleotides recognizable and cleavable by the RNase at the 5′-end (such as at least 7 such nucleotides), followed by a contiguous stretch of non-RNase recruiting nucleotide analogues towards the 3′-end. Other chimeras according to the invention, called mixmers consisting of an alternate composition of DNA or modified nucleotides recognizable and cleavable by RNase and non-RNase recruiting nucleotide analogues. Some nucleotide analogues may also be able to mediate RNaseH binding and cleavage. Since α-L-LNA recruits RNaseH activity to a certain extent, smaller gaps of DNA or modified nucleotides recognizable and cleavable by the RNaseH for the gapmer construct might be required, and more flexibility in the mixmer construction might be introduced.

### Gapmer Design

Preferably, the oligomer of the invention is a gapmer. A gapmer oligomer is an oligomer which comprises a contiguous stretch of nucleotides which is capable of recruiting an RNAse, such as RNAseH, such as a region of at least 6 or 7 DNA nucleotides, referred to herein in as region B, wherein region B is flanked both 5′ and 3′ by regions of affinity enhancing nucleotide analogues, such as between 1-6 nucleotide analogues 5′ and 3′ to the contiguous stretch of nucleotides which is capable of recruiting RNAse—these regions are referred to as regions A and C respectively.

In some embodiments, the nucleotides which are capable of recruiting RNAse are selected from the group consisting of DNA nucleotides, alpha-L-LNA nucleotides, C4′ alkylayted DNA. (see PCT/EP2009/050349 hereby incorporated by reference), and UNA nucleotides (see Fluiter et al., Mol. Biosyst., 2009, 10, 1039 hereby incorporated by reference). In some embodiments, region B consists of a contiguous length of at least 6 or 7 DNA nucleotides, or nucleotides selected from the group consisting of DNA and alpha-L-LNA.

Preferably the gapmer comprises a (poly)nucleotide sequence of formula (5′ to 3′), A-B-C, or optionally A-B-C-D or D-A-B-C, wherein; region A (5′ region) consists or comprises of at least one nucleotide analogue, such as at least one LNA unit, such as between 1-6 nucleotide analogues, such as LNA units, and; region B consists or comprises of at least five consecutive nucleotides which are capable of recruiting RNAse (when formed in a duplex with a complementary RNA molecule, such as the mRNA target), such as DNA nucleotides, and; region C (3′ region) consists or comprises of at least one nucleotide analogue, such as at least one LNA unit, such as between 1-6 nucleotide analogues, such as LNA units, and; region D, when present consists or comprises of 1, 2 or 3 nucleotide units, such as DNA nucleotides.

In various embodiments, region A consists of 1, 2, 3, 4, 5 or 6 nucleotide analogues, such as LNA units, such as between 2-5 nucleotide analogues, such as 2-5 LNA units, such as 3 or 4 nucleotide analogues, such as 3 or 4 LNA units; and/or region C consists of 1, 2, 3, 4, 5 or 6 nucleotide analogues, such as LNA units, such as between 2-5 nucleotide analogues, such as 2-5 LNA units, such as 3 or 4 nucleotide analogues, such as 3 or 4 LNA units.

In various embodiments B consists or comprises of 5, 6, 7, 8, 9, 10, 11 or 12 consecutive nucleotides which are capable of recruiting RNAse, or between 6-10, or between 7-9, such as 8 consecutive nucleotides which are capable of recruiting RNAse. In various embodiments region B consists or comprises at least one DNA nucleotide unit, such as 1-12 DNA units, preferably between 4-12 DNA units, more preferably between 6-10 DNA units, such as between 7-10 DNA units, most preferably 8, 9 or 10 DNA units.

In various embodiments region A consist of 3 or 4 nucleotide analogues, such as LNA, region B consists of 7, 8, 9 or 10 DNA units, and region C consists of 3 or 4 nucleotide analogues, such as LNA. Such designs include (A-B-C) 3-10-3, 3-10-4, 4-10-3, 3-9-3, 3-9-4, 4-9-3, 3-8-3, 3-8-4, 4-8-3, 3-7-3, 3-7-4, 4-7-3, and may further include region D, which may have one or 2 nucleotide units, such as DNA units.

Further gapmer designs are disclosed in WO2004/046160 and are hereby incorporated by reference.

US provisional application, 60/977,409, hereby incorporated by reference, refers to ‘shortmer’ gapmer oligomers, which, in various embodiments may be the gapmer oligomer according to the present invention.

In various embodiments the oligomer is consisting of a contiguous nucleotide sequence of a total of 10, 11, 12, 13 or 14 nucleotide units, wherein the contiguous nucleotide sequence is of formula (5′-3′), A-B-C, or optionally A-B-C-D or D-A-B-C, wherein; A consists of 1, 2 or 3 nucleotide analogue units, such as LNA units; B consists of 7, 8 or 9 contiguous nucleotide units which are capable of recruiting RNAse when formed in a duplex with a complementary RNA molecule (such as a mRNA target); and C consists of 1, 2 or 3 nucleotide analogue units, such as LNA units. When present, D consists of a single DNA unit.

In various embodiments A consists of 1 LNA unit. In various embodiments A consists of 2 LNA units. In various embodiments A consists of 3 LNA units. In various embodiments C consists of 1 LNA unit. In various embodiments C consists of 2 LNA units. In various embodiments C consists of 3 LNA units. In various embodiments B consists of 7 nucleotide units. In various embodiments B consists of 8 nucleotide units. In various embodiments B consists of 9 nucleotide units. In various embodiments B comprises of between 1-9 DNA units, such as 2, 3, 4, 5, 6, 7 or 8 DNA units. In various embodiments B consists of DNA units. In various embodiments B comprises of at least one LNA unit which is in the alpha-L configuration, such as 2, 3, 4, 5, 6, 7, 8 or 9 LNA units in the alpha-L-configuration. In various embodiments B comprises of at least one alpha-L-oxy LNA unit or wherein all the LNA units in the alpha-L-configuration are alpha-L-oxy LNA units. In various embodiments the number of nucleotides present in A-B-C are selected from the group consisting of (nucleotide analogue units—region B—nucleotide analogue units): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, 3-10-1. In various embodiments the number of nucleotides in A-B-C are selected from the group consisting of: 2-7-1, 1-7-2, 2-7-2, 3-7-3, 2-7-3, 3-7-2, 3-7-4, and 4-7-3. In various embodiments both A and C consists of two LNA units each, and B consists of 8 or 9 nucleotide units, preferably DNA units.

### Internucleotide Linkages

The terms “linkage group” or “internucleotide linkage” are intended to mean a group capable of covalently coupling together two nucleotides. Specific and preferred examples include phosphate groups and phosphorothioate groups.

The nucleotides of the oligomer of the invention or contiguous nucleotides sequence thereof are coupled together via linkage groups. Suitably each nucleotide is linked to the 3′ adjacent nucleotide via a linkage group.

Suitable internucleotide linkages include those listed within PCT/DK2006/000512, for example the internucleotide linkages listed on the first paragraph of page 34 of PCT/DK2006/000512 (hereby incorporated by reference).

It is, in various embodiments, preferred to modify the internucleotide linkage from its normal phosphodiester to one that is more resistant to nuclease attack, such as phosphorothioate or boranophosphate—these two, being cleavable by RNase H, also allow that route of antisense inhibition in reducing the expression of the target gene.

Suitable sulphur (S) containing internucleotide linkages as provided herein may be preferred. Phosphorothioate internucleotide linkages are also preferred, particularly for the gap region (B) of gapmers. Phosphorothioate linkages may also be used for the flanking regions (A and C, and for linking A or C to D, and within region D, as appropriate).

Regions A, B and C, may however comprise internucleotide linkages other than phosphorothioate, such as phosphodiester linkages, particularly, for instance when the use of nucleotide analogues protects the internucleotide linkages within regions A and C from endo-nuclease degradation—such as when regions A and C comprise LNA nucleotides.

The internucleotide linkages in the oligomer may be phosphodiester, phosphorothioate or boranophosphate so as to allow RNase H cleavage of targeted RNA. Phosphorothioate is preferred, for improved nuclease resistance and other reasons, such as ease of manufacture.

In one aspect of the oligomer of the invention, the nucleotides and/or nucleotide analogues are linked to each other by means of phosphorothioate groups.

It is recognised that the inclusion of phosphodiester linkages, such as one or two linkages, into an otherwise phosphorothioate oligomer, particularly between or adjacent to nucleotide analogue units (typically in region A and or C) can modify the bioavailability and/or bio-distribution of an oligomer—see WO2008/053314, hereby incorporated by reference.

In some embodiments, such as the embodiments referred to above, where suitable and not specifically indicated, all remaining linkage groups are either phosphodiester or phosphorothioate, or a mixture thereof.

In some embodiments all the internucleotide linkage groups are phosphorothioate.

When referring to specific gapmer oligonucleotide sequences, such as those provided herein it will be understood that, in various embodiments, when the linkages are phosphorothioate linkages, alternative linkages, such as those disclosed herein may be used, for example phosphate (phosphodiester) linkages may be used, particularly for linkages between nucleotide analogues, such as LNA, units. Likewise, when referring to specific gapmer oligonucleotide sequences, such as those provided herein, when the C residues are annotated as 5′ methyl modified cytosine, in various embodiments, one or more of the Cs present in the oligomer may be unmodified C residues.

### Oligomeric Compounds

The oligomers of the invention may, for example, be a gapmer with a structure according to a sequence selected from the group consisting of SEQ ID NO 62-72, such as SEQ ID NO 62, 63, 64 or 65, or SEQ ID NO 66, 67, 68, 69, 70, 71 and 72.

The oligomers of the invention may, for example, be selected from the group consisting of: SEQ ID NOs 29-50, such as SEQ ID NOs 29, 30, 31 and 32, or SEQ ID NOs 33, 34, 35, 36, 37, 38 and 39.

### Conjugates

In the context the term “conjugate” is intended to indicate a heterogenous molecule formed by the covalent attachment (“conjugation”) of the oligomer as described herein to one or more non-nucleotide, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol.

Therefore, in various embodiments, the oligomer of the invention may comprise both a polynucleotide region which typically consists of a contiguous sequence of nucleotides, and a further non-nucleotide region. When referring to the oligomer of the invention consisting of a contiguous nucleotide sequence, the compound may comprise non-nucleotide components, such as a conjugate component.

In various embodiments of the invention the oligomeric compound is linked to ligands/conjugates, which may be used, e.g. to increase the cellular uptake of oligomeric compounds. WO2007/031091 provides suitable ligands and conjugates, which are hereby incorporated by reference.

The invention also provides for a conjugate comprising the compound according to the invention as herein described, and at least one non-nucleotide or non-polynucleotide moiety covalently attached to said compound. Therefore, in various embodiments where the compound of the invention consists of a specified nucleic acid or nucleotide sequence, as herein disclosed, the compound may also comprise at least one non-nucleotide or non-polynucleotide moiety (e.g. not comprising one or more nucleotides or nucleotide analogues) covalently attached to said compound.

Conjugation (to a conjugate moiety) may enhance the activity, cellular distribution or cellular uptake of the oligomer of the invention. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

The oligomers of the invention may also be conjugated to active drug substances, for example, aspirin, ibuprofen, a sulfa drug, an antidiabetic, an antibacterial or an antibiotic.

In certain embodiments the conjugated moiety is a sterol, such as cholesterol.

In various embodiments, the conjugated moiety comprises or consists of a positively charged polymer, such as a positively charged peptides of, for example between 1-50, such as 2-20 such as 3-10 amino acid residues in length, and/or polyalkylene oxide such as polyethylglycol(PEG) or polypropylene glycol—see WO 2008/034123, hereby incorporated by reference. Suitably the positively charged polymer, such as a polyalkylene oxide may be attached to the oligomer of the invention via a linker such as the releasable inker described in WO 2008/034123.

By way of example, the following conjugate moieties may be used in the conjugates of the invention:

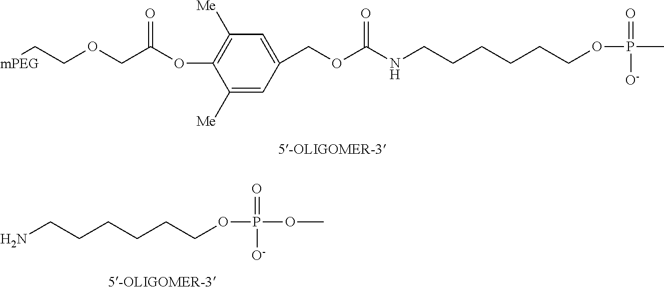

### Activated Oligomers

The term “activated oligomer,” as used herein, refers to an oligomer of the invention that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that is preferably hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl. In some embodiments, the functional moiety is self-cleaving. In other embodiments, the functional moiety is biodegradable. See e.g., U.S. Pat. No. 7,087,229, which is incorporated by reference herein in its entirety.

In some embodiments, oligomers of the invention are functionalized at the 5′ end in order to allow covalent attachment of the conjugated moiety to the 5′ end of the oligomer. In other embodiments, oligomers of the invention can be functionalized at the 3′ end. In still other embodiments, oligomers of the invention can be functionalized along the backbone or on the heterocyclic base moiety. In yet other embodiments, oligomers of the invention can be functionalized at more than one position independently selected from the 5′ end, the 3′ end, the backbone and the base.

In some embodiments, activated oligomers of the invention are synthesized by incorporating during the synthesis one or more monomers that is covalently attached to a functional moiety. In other embodiments, activated oligomers of the invention are synthesized with monomers that have not been functionalized, and the oligomer is functionalized upon completion of synthesis. In some embodiments, the oligomers are functionalized with a hindered ester containing an aminoalkyl linker, wherein the alkyl portion has the formula (CH₂)_{w}, wherein w is an integer ranging from 1 to 10, preferably about 6, wherein the alkyl portion of the alkylamino group can be straight chain or branched chain, and wherein the functional group is attached to the oligomer via an ester group (—O—C(O)—(CH₂)_{w}NH).

In other embodiments, the oligomers are functionalized with a hindered ester containing a (CH₂)_{w}-sulfhydryl (SH) linker, wherein w is an integer ranging from 1 to 10, preferably about 6, wherein the alkyl portion of the alkylamino group can be straight chain or branched chain, and wherein the functional group attached to the oligomer via an ester group (—O—C(O)—(CH₂)_{w}SH)

In some embodiments, sulfhydryl-activated oligonucleotides are conjugated with polymer moieties such as polyethylene glycol or peptides (via formation of a disulfide bond).

Activated oligomers containing hindered esters as described above can be synthesized by any method known in the art, and in particular by methods disclosed in PCT Publication No. WO 2008/034122 and the examples therein, which is incorporated herein by reference in its entirety.

In still other embodiments, the oligomers of the invention are functionalized by introducing sulfhydryl, amino or hydroxyl groups into the oligomer by means of a functionalizing reagent substantially as described in U.S. Pat. Nos. 4,962,029 and 4,914,210, i.e., a substantially linear reagent having a phosphoramidite at one end linked through a hydrophilic spacer chain to the opposing end which comprises a protected or unprotected sulfhydryl, amino or hydroxyl group. Such reagents primarily react with hydroxyl groups of the oligomer. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers of the invention have a functionalizing reagent coupled to a hydroxyl group on the backbone of the oligomer. In yet further embodiments, the oligomer of the invention is functionalized with more than one of the functionalizing reagents as described in U.S. Pat. Nos. 4,962,029 and 4,914,210, incorporated herein by reference in their entirety. Methods of synthesizing such functionalizing reagents and incorporating them into monomers or oligomers are disclosed in U.S. Pat. Nos. 4,962,029 and 4,914,210.

In some embodiments, the 5′-terminus of a solid-phase bound oligomer is functionalized with a dienyl phosphoramidite derivative, followed by conjugation of the deprotected oligomer with, e.g., an amino acid or peptide via a Diels-Alder cycloaddition reaction.

In various embodiments, the incorporation of monomers containing 2′-sugar modifications, such as a 2′-carbamate substituted sugar or a 2′-(O-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al., Tetrahedron Letters, 1991, 34, 7171.

In still further embodiments, the oligomers of the invention may have amine-containing functional moieties on the nucleobase, including on the N6 purine amino groups, on the exocyclic N2 of guanine, or on the N4 or 5 positions of cytosine. In various embodiments, such functionalization may be achieved by using a commercial reagent that is already functionalized in the oligomer synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.).

### Compositions

The oligomer of the invention may be used in pharmaceutical formulations and compositions. Suitably, such compositions comprise a pharmaceutically acceptable diluent, carrier, salt or adjuvant. PCT/DK2006/000512 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in PCT/DK2006/000512—which are also hereby incorporated by reference.

### Applications

The oligomers of the invention may be utilized as research reagents for, for example, diagnostics, therapeutics and prophylaxis.

In research, such oligomers may be used to specifically inhibit the synthesis of Hsp70-2 protein (typically by degrading or inhibiting the mRNA and thereby prevent protein formation) in cells and experimental animals thereby facilitating functional analysis of the target or an appraisal of its usefulness as a target for therapeutic intervention.

In diagnostics the oligomers may be used to detect and quantitate Hsp70-2 expression in cell and tissues by northern blotting, in-situ hybridisation or similar techniques.

For therapeutics, an animal or a human, suspected of having a disease or disorder, which can be treated by modulating the expression of Hsp70-2 is treated by administering oligomeric compounds in accordance with this invention. Further provided are methods of treating a mammal, such as treating a human, suspected of having or being prone to a disease or condition, associated with expression of Hsp70-2 by administering a therapeutically or prophylactically effective amount of one or more of the oligomers or compositions of the invention.

The invention also provides for the use of the compound or conjugate of the invention as described for the manufacture of a medicament for the treatment of a disorder as referred to herein, or for a method of the treatment of as a disorder as referred to herein.

The invention also provides for a method for treating a disorder as referred to herein said method comprising administering a compound according to the invention as herein described, and/or a conjugate according to the invention, and/or a pharmaceutical composition according to the invention to a patient in need thereof.

### Medical Indications

The down-regulation of Hsp70-2 (and optionally Hsp70-1) is considered to be beneficial for the treatment of hyperproliferative diseases such as cancer. In the context of the present invention, in various embodiments the oligomer targets both Hsp70-1 and Hsp70-2, and it is considered that oligomers which target both Hsp70-2 and Hsp70-2 may in some embodiments, be particularly effective in treatment of hyperproliferative diseases such as cancer. Whilst it is considered that cancer cells are particularly (preferentially) sensitive to inhibition of Hsp70-2 (and optionally Hsp70-1), and as such the use of the oligomers of the invention may be used to target cancer cells per se, it is also considered that the treatment may be used for cancer types where the levels of Hsp70-2 (and optionally Hsp70-1) are elevated (i.e. over-expressed).

The oligomers and other compositions according to the invention can be used for the treatment of conditions associated with over expression or expression of Hsp70-2 and/or Hsp70-1

The invention further provides use of a compound of the invention in the manufacture of a medicament for the treatment of a disease, disorder or condition as referred to herein.

Generally stated, one aspect of the invention is directed to a method of treating a mammal suffering from or susceptible to conditions associated with abnormal level, such as over-expression, of Hsp70-2 and/or Hsp70-1, comprising administering to the mammal and therapeutically effective amount of an oligomer targeted to Hsp70-2 that comprises one or more LNA units.

An interesting aspect of the invention is directed to the use of an oligomer (compound) as defined herein or a conjugate as defined herein for the preparation of a medicament for the treatment of a disease, disorder or condition as referred to herein.

The methods of the invention may in some embodiments be employed for treatment or prophylaxis against diseases caused by abnormal levels of Hsp70-2.

Alternatively stated, in some embodiments, the invention is furthermore directed to a method for treating abnormal levels, such as over-expression, of Hsp70-2, said method comprising administering a oligomer of the invention, or a conjugate of the invention or a pharmaceutical composition of the invention to a patient in need thereof.

The invention also relates to an oligomer, a composition or a conjugate as defined herein for use as a medicament.

The invention further relates to use of a compound, composition, or a conjugate as defined herein for the manufacture of a medicament for the treatment of abnormal levels, such as over-espression, of Hsp70-2 or expression of mutant forms of Hsp70-2 (such as allelic variants, such as those associated with one of the diseases referred to herein).

Moreover, the invention relates to a method of treating a subject suffering from a disease or condition such as those referred to herein.

A patient who is in need of treatment is a patient suffering from or likely to suffer from the disease or disorder.

In various embodiments, the term ‘treatment’ as used herein refers to both treatment of an existing disease (e.g. a disease or disorder as herein referred to), or prevention of a disease, i.e. prophylaxis. It will therefore be recognised that treatment as referred to herein may, in various embodiments, be prophylactic.

Embodiments. The invention, in some aspects, may include one or more of the following embodiments:

1. An oligomer of between 10-30 nucleotides in length which comprises a contiguous nucleotide sequence of a total of between 10-30 nucleotides, wherein said contiguous nucleotide sequence is at least 80% homologous to a region corresponding to a mammalian Hsp70-2 gene or mRNA, such as SEQ ID NO: 1 or naturally occurring variant thereof.
2. The oligomer according to embodiment 1, wherein the contiguous nucleotide sequence is at least 80% homologous to a region corresponding to 7-28 and 40-50.
3. The oligomer according to embodiment 1 or 2, wherein the contiguous nucleotide sequence comprises no mismatches or no more than one or two mismatches with the corresponding region of 1.
4. The oligomer according to any one of embodiments 1-3, wherein the nucleotide sequence of the oligomer consists of the contiguous nucleotide sequence.
5. The oligomer according to any one of embodiments 1-4, wherein the contiguous nucleotide sequence is between 10-18 nucleotides in length.
6. The oligomer according to any one of embodiments 1-5, wherein the contiguous nucleotide sequence comprises nucleotide analogues.
7. The oligomer according to embodiment 6, wherein the nucleotide analogues are sugar modified nucleotides, such as sugar modified nucleotides selected from the group consisting of: Locked Nucleic Acid (LNA) units; 2′-O-alkyl-RNA units, 2′-OMe-RNA units, 2′-amino-DNA units, and 2′-fluoro-DNA units.
8. The oligomer according to embodiment 7, wherein the nucleotide analogues are LNA.
9. The oligomer according to any one of embodiments 6-8 which is a gapmer.
10. The oligomer according to any one of embodiments 1-9, which inhibits the expression of Hsp70-2 gene or mRNA in a cell which is expressing Hsp70-2 gene or mRNA.
11. A conjugate comprising the oligomer according to any one of embodiments 1-10, and at least one non-nucleotide or non-polynucleotide moiety covalently attached to said oligomer.
12. A pharmaceutical composition comprising the oligomer according to any one of embodiments 1-10, or the conjugate according to embodiment 11, and a pharmaceutically acceptable diluent, carrier, salt or adjuvant.
13. The oligomer according to any one of embodiments 1-10, or the conjugate according to embodiment 11, for use as a medicament, such as for the treatment of hyperproliferative diseases, such as cancer.
14. The use of an oligomer according to any one of the embodiments 1-10, or a conjugate as defined in embodiment 11, for the manufacture of a medicament for the treatment of hyperproliferative diseases, such as cancer.
15. A method of treating hyperproliferative diseases, such as cancer, said method comprising administering an oligomer according to any one of the embodiments 1-10, or a conjugate according to embodiment 11, or a pharmaceutical composition according to embodiment 12, to a patient suffering from, or likely to suffer from hyperproliferative diseases, such as cancer.
16. A method for the inhibition of Hsp70-2 in a cell which is expressing Hsp70-2, said method comprising administering an oligomer according to any one of the embodiments 1-10, or a conjugate according to embodiment 11 to said cell so as to inhibit Hsp70-2 in said cell.
17. A method for the simultaneous inhibition of Hsp70-2 and Hsp70-1 in a cell which is expressing both Hsp70-2 and Hsp70-1, said method comprising administering an oligomer, or a conjugate according to the invention to said cell so as to effect the inhibition of Hsp70-2 and Hsp70-1 in said cell.

### EXAMPLES

### Example 1

### Monomer Synthesis

The LNA monomer building blocks and derivatives were prepared following published procedures and references—see WO07/031,081 and the references cited therein.

### Example 2

### Oligonucleotide Synthesis

Oligonucleotides were synthesized according to the method described in WO07/031,081. Table 1 shows examples of antisense oligonucleotide sequences of the invention. Tables 2 and 3 show examples of antisense oligonucleotides (oligos) of the invention.

### Example 3

### Design of the Oligonucleotides

In accordance with the present invention, a series of different oligonucleotides were designed to target different regions of human Hsp70-2 (HSPA2) (GenBank accession number NM_{—}021979, SEQ ID NO: 1). Some oligonucleotides were designed to also target regions of HSPA1A and HSPA1B (GenBank accession number NM_{—}005345 and NM_{—}005346, SEQ ID NO: 2 and SEQ ID NO 3) with one mismatching base. All oligonucleotides were designed to have a minimum of 3 mismatching bases toward HSPA5 (GenBank accession number NM_{—}005347, SEQ ID NO: 4), HSPA8 (GenBank accession number NM_{—}006597, SEQ ID NO: 5) and HSPA9 (GenBank accession number NM_{—}004134, SEQ ID NO: 6).

TABLE 1a and b
Antisense oligonucleotide sequences of the invention

| SEQ ID NO | Sequence (5′-3′) | Length (bases) | Target site |
|---|---|---|---|
| SEQ ID NO: 7 | ATCTCCACCTTGCCAT | 16 | 453-468 |
| SEQ ID NO: 8 | ACCTCCTGACACTTGT | 16 | 2106-2121 |
| SEQ ID NO: 9 | CCACCTTGCCATGTTG | 16 | 449-464 |
| SEQ ID NO: 10 | TCCACCTTGCCATGTT | 16 | 450-465 |
| SEQ ID NO: 11 | TGCTTGATGTTGTAGG | 16 | 2028-2043 |
| SEQ ID NO: 12 | CACCTCCTGACACTTG | 16 | 2107-2122 |
| SEQ ID NO: 13 | TGGCACAAGGACATTT | 16 | 2372-2387 |
| SEQ ID NO: 14 | ACTAAGTTGTTGCACC | 16 | 2438-2453 |
| SEQ ID NO: 15 | ATTAAAGAGAAACCTC | 16 | 2498-2513 |
| SEQ ID NO: 16 | TTTCAGCTTTACTTTA | 16 | 2744-2759 |
| SEQ ID NO: 17 | CAATTTCAGCTTTACT | 16 | 2747-2762 |
| SEQ ID NO: 18 | ATCTCCACCTTGCCA | 15 | 454-468 |
| SEQ ID NO: 19 | ACCTCCTGACACTT | 14 | 2108-2121 |
| SEQ ID NO: 20 | ACCTCCTGACACT | 13 | 2109-2121 |
| SEQ ID NO: 21 | ACCTTGCCATGTTG | 14 | 449-462 |
| SEQ ID NO: 22 | CCACCTTGCCATGT | 14 | 451-464 |
| SEQ ID NO: 23 | TAAGTTGTTGCACC | 14 | 2438-2451 |
| SEQ ID NO: 24 | AAGTTGTTGCACC | 13 | 2438-2450 |
| SEQ ID NO: 25 | TAAGTTGTTGCAC | 13 | 2439-2451 |
| SEQ ID NO: 26 | CTAAGTTGTTGCAC | 14 | 2439-2452 |
| SEQ ID NO: 27 | ACTAAGTTGTTGCA | 14 | 2440-2453 |
| SEQ ID NO: 28 | TCAGCTTTACTTTA | 14 | 2744-2757 |

| 16mer SEQ ID NO | 24mer SEQ ID NO | Sequence (5′-3′) |
|---|---|---|
| SEQ ID NO: 7 | SEQ ID NO: 40 | GATGATCTCCACCTTGCCATGTTG |
| SEQ ID NO: 8 | SEQ ID NO: 41 | GATCACCTCCTGACACTTGTCGAG |
| SEQ ID NO: 9 | SEQ ID NO: 42 | ATCTCCACCTTGCCATGTTGGAAG |
| SEQ ID NO: 10 | SEQ ID NO: 43 | GATCTCCACCTTGCCATGTTGGAA |
| SEQ ID NO: 11 | SEQ ID NO: 44 | CGTCTGCTTGATGTTGTAGGTATA |
| SEQ ID NO: 12 | SEQ ID NO: 45 | TGATCACCTCCTGACACTTGTCGA |
| SEQ ID NO: 13 | SEQ ID NO: 46 | GTACTGGCACAAGGACATTTCAAA |
| SEQ ID NO: 14 | SEQ ID NO: 47 | TTAAACTAAGTTGTTGCACCTCTC |
| SEQ ID NO: 15 | SEQ ID NO: 48 | ATGCATTAAAGAGAAACCTCGAAT |
| SEQ ID NO: 16 | SEQ ID NO: 49 | ATCATTTCAGCTTTACTTTACATT |
| SEQ ID NO: 17 | SEQ ID NO: 50 | AGATCAATTTCAGCTTTACTTTAC |

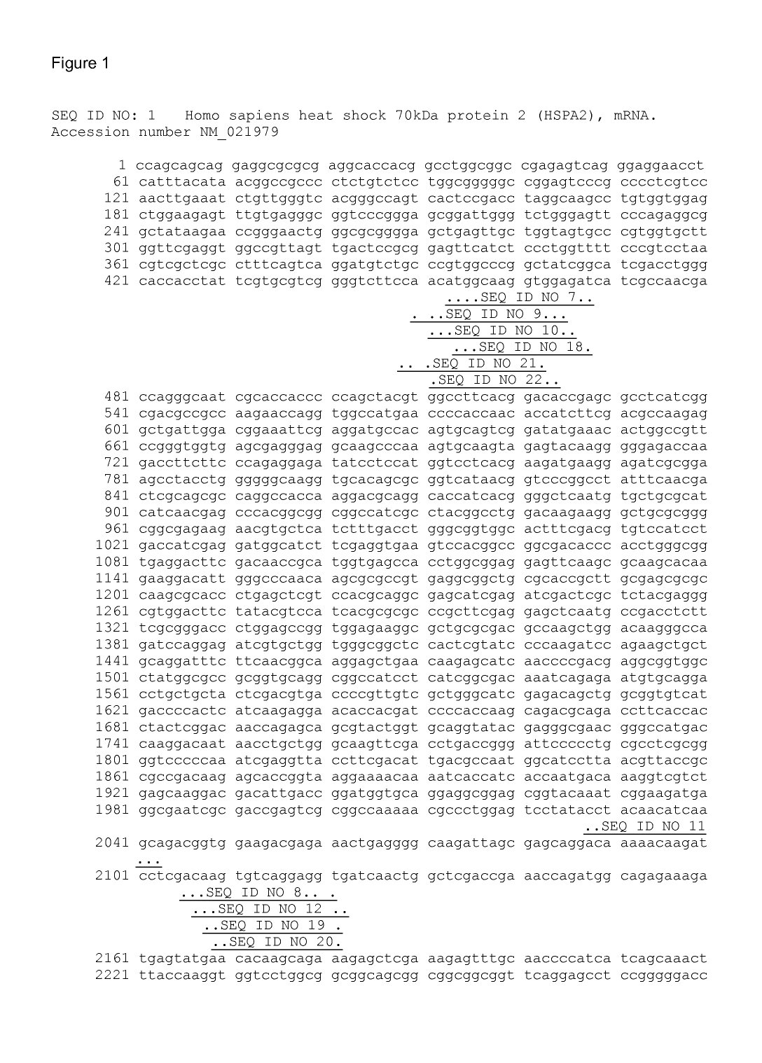

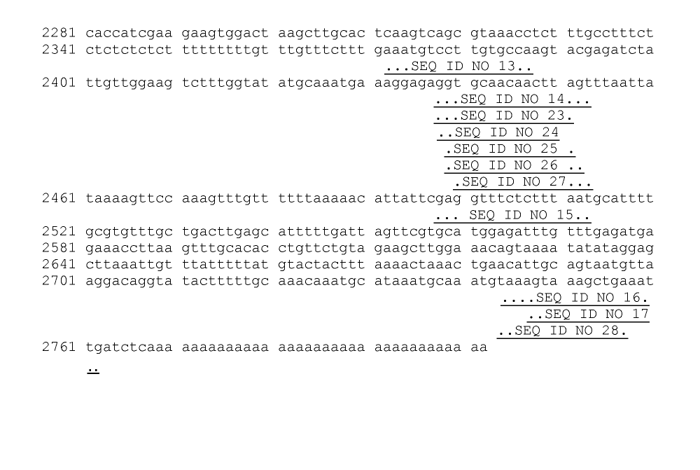

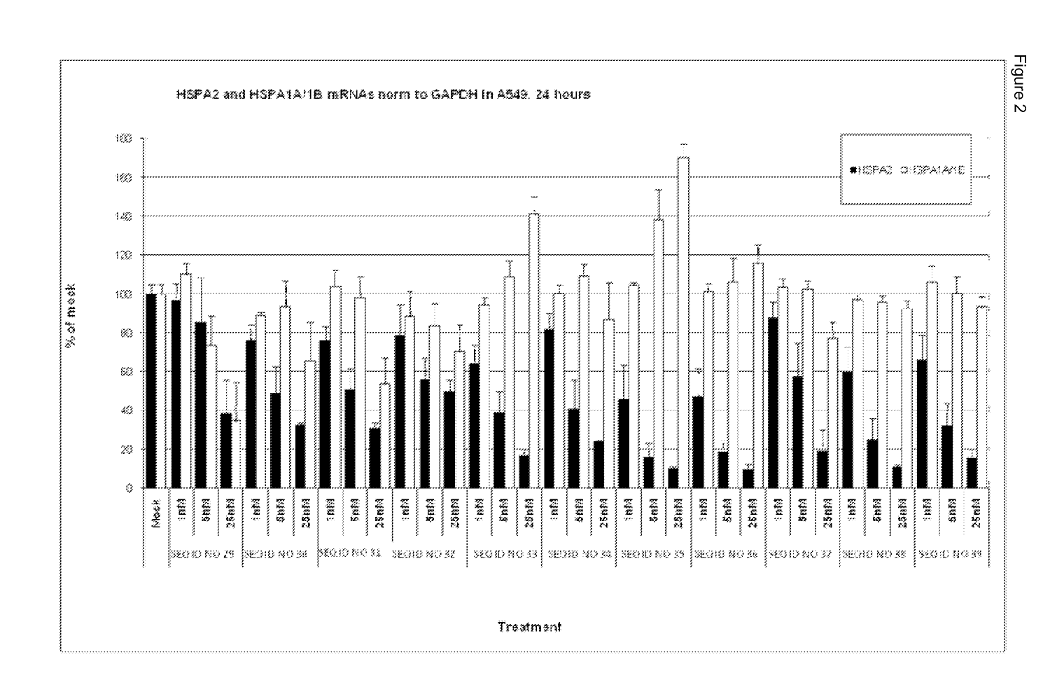

SEQ ID NOS: are oligo sequences designed to target human
Hsp70-2 (HSPA2) SEQ ID NO 1 (See FIG. 1).
SEQ ID NOS: 7, 8, 9, 10, 18, 19, 20, 21 & 22 are
oligonucleotides designed to target HSPA1A and HSPA1B with
one mismatching base.

TABLE 2
Oligonucleotide Designs of the invention

| SEQ ID NO | Sequence (5′-3′) |
|---|---|
| SEQ ID NO: 51 | ATCtccaccttgcCAT |
| SEQ ID NO: 52 | ACCtcctgacactTGT |
| SEQ ID NO: 53 | CCAccttgccatgTTG |
| SEQ ID NO: 54 | TCCaccttgccatGTT |
| SEQ ID NO: 55 | TGCttgatgttgtAGG |
| SEQ ID NO: 56 | CACctcctgacacTTG |
| SEQ ID NO: 57 | TGGcacaaggacaTTT |
| SEQ ID NO: 58 | ACTaagttgttgcACC |
| SEQ ID NO: 59 | ATTaaagagaaacCTC |
| SEQ ID NO: 60 | TTTcagctttactTTA |
| SEQ ID NO: 61 | CAAtttcagctttACT |
| SEQ ID NO: 62 | ATCtccaccttgCCA |
| SEQ ID NO: 63 | ACCtcctgacaCTT |
| SEQ ID NO: 64 | ACctcctgacACT |
| SEQ ID NO: 65 | ACCttgccatgTTG |
| SEQ ID NO: 66 | CCAccttgccaTGT |
| SEQ ID NO: 67 | TAAgttgttgcACC |
| SEQ ID NO: 68 | AAgttgttgcACC |
| SEQ ID NO: 69 | TAagttgttgCAC |
| SEQ ID NO: 70 | CTAagttgttgCAC |
| SEQ ID NO: 71 | ACTaagttgttGCA |
| SEQ ID NO: 72 | TCAgctttactTTA |

In SEQ ID NOs: 51-72, upper case
letters indicate nucleotide analogues, such as
LNA units, such as oxy-LNA, such as beta-D-oxy
LNA. Lower case letters represent nucleotide
units. Internucleotide linkages may be, for
example, phosphorothiote linkage or phosphodiester
linkage, or combination thereof. LNA cytosines
are preferably 5′methyl cytosine.

### Example 4

### In Vitro Model: Cell Culture

The effect of antisense oligonucleotides on target nucleic acid expression can be tested in any of a variety of cell types provided that the target nucleic acid is present at measurable levels. The target can be expressed endogenously or by transient or stable transfection of a nucleic acid encoding said target. The expression level of target nucleic acid can be routinely determined using, for example, Northern blot analysis, Real-Time PCR, Ribonuclease protection assays. The following cell types are provided for illustrative purposes, but other cell types can be routinely used, provided that the target is expressed in the cell type chosen.

Cells were cultured in the appropriate medium as described below and maintained at 37° C. at 95-98% humidity and 5% CO₂. Cells were routinely passaged 2-3 times weekly.

A549: The human prostate cancer cell line A549 was cultured in DMEM (Sigma)+10% fetal bovine serum (FBS)+2 mM Glutamax I+gentamicin (25 μg/ml).

### Example 5

### In Vitro Model: Treatment with Antisense Oligonucleotide

The cell lines listed in example 4 were treated with oligonucleotide using the cationic liposome formulation LipofectAMINE 2000 (Gibco) as transfection vehicle. Cells were seeded in 6-well cell culture plates (NUNC) and treated when 80-90% confluent. Oligo concentrations used ranged from 1 nM to 25 nM final concentration. Formulation of oligo-lipid complexes were carried out essentially as described by the manufacturer using serum-free OptiMEM (Gibco) and a final lipid concentration of 5 μg/mL LipofectAMINE 2000. Cells were incubated at 37° C. for 4 hours and treatment was stopped by removal of oligo-containing culture medium. Cells were washed and serum-containing media was added. After oligo treatment cells were allowed to recover for 20 hours before they were harvested for RNA analysis.

### Example 6

### In Vitro Model: Extraction of RNA and cDNA Synthesis

### Total RNA Isolation and First Strand Synthesis

Total RNA was extracted from cells transfected as described above and using the Qiagen RNeasy kit (Qiagen cat. no. 74104) according to the manufacturer's instructions. First strand synthesis was performed using Reverse Transcriptase reagents from Ambion according to the manufacturer's instructions.

For each sample 0.5 μg total RNA was adjusted to (10.8 μl) with RNase free H₂O and mixed with 2 μl random decamers (50 μM) and 4 μl dNTP mix (2.5 mM each dNTP) and heated to 70° C. for 3 min after which the samples were rapidly cooled on ice. After cooling the samples on ice, 2 μl 10× Buffer RT, 1 μl MMLV Reverse Transcriptase (100 U/μl) and 0.25 μl RNase inhibitor (10 U/μl) was added to each sample, followed by incubation at 42° C. for 60 min, heat inactivation of the enzyme at 95° C. for 10 min and then the sample was cooled to 4° C.

### Example 7

### In Vitro Model: Analysis of Oligonucleotide Inhibition of HSPA2 and HSPA1A/1E3 Expression by Real-Time PCR

Antisense modulation of HSPA2 and HSPA1/1B expression can be assayed in a variety of ways known in the art. For example, HSPA2 mRNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA.

Methods of RNA isolation and RNA analysis such as Northern blot analysis is routine in the art and is taught in, for example, Current Protocols in Molecular Biology, John Wiley and Sons.

Real-time quantitative (PCR) can be conveniently accomplished using the commercially available Multi-Color Real Time PCR Detection System, available from Applied Biosystem.

Real-time Quantitative PCR Analysis of HSPA2 and HSPA1A Plus HSPA1 B mRNA Levels

The sample content of human HSPA2 mRNA was quantified using the human HSPA2 ABI Prism Pre-Developed TaqMan Assay Reagents (Applied Biosystems cat. no. Hs00706997_s1 according to the manufacturer's instructions.

The sample content of human HSPA1A plus HSPA1B mRNA was quantified using the human HSPA1A/1B ABI Prism Pre-Developed TaqMan Assay Reagents (Applied Biosystems cat. no. Hs00271229_s1 according to the manufacturer's instructions.

Glyceraldehyde-3-phosphate dehydrogenase (GAPDH) mRNA quantity was used as an endogenous control for normalizing any variance in sample preparation.

The sample content of human GAPDH mRNA was quantified using the human GAPDH ABI Prism Pre-Developed TaqMan Assay Reagent (Applied Biosystems cat. no. 4310884E) according to the manufacturer's instructions.

Real-time Quantitative PCR is a technique well known in the art and is taught in for example Heid et al. Real time quantitative PCR, Genome Research (1996), 6: 986-994.

### Real Time PCR

The cDNA from the first strand synthesis performed as described in example 5 was diluted 2-20 times, and analyzed by real time quantitative PCR using Taqman 7500 FAST or 7900 FAST from Applied Biosystems. The primers and probe were mixed with 2× Taqman Fast Universal PCR master mix (2×) (Applied Biosystems Cat. # 4364103) and added to 4 μl cDNA to a final volume of 10 μl. Each sample was analysed in duplicate. Assaying 2 fold dilutions of a cDNA that had been prepared on material purified from a cell line expressing the RNA of interest generated standard curves for the assays. Sterile H₂O was used instead of cDNA for the no template control. PCR program: 60° C. for 2 minutes, then 95° C. for 30 seconds, followed by 40 cycles of 95° C., 3 seconds, 60° C., 20-30 seconds. Relative quantities of target mRNA sequence were determined from the calculated Threshold cycle using the Applied Biosystems Fast System SDS Software Version 1.3.1.21. or SDS Software Version 2.3.

### Example 8

### In Vitro Analysis: Antisense Inhibition of Human HSPA2 and HSPA1A/1B Expression by Oligonucleotide Compounds

Oligonucleotides presented in Table 3 were evaluated for their potential to knockdown of HSPA2 and HSPA1A/1B at concentrations of 1, 5 and 25 nM (see FIG. 1).

TABLE 3
Antisense Inhibition of Human HSPA2 and
HSPA1A/1B expression by oligonucleotides.

| | | % inhibition | |
|---|---|---|---|
| Test | | of | % inhibition |
| substance | Sequence (5′-3′) | HSPA1A/1B | of HSPA2 |
| SEQ ID NO: 29 | AsTsCstₛcscₛaₛcₛcₛtₛtₛgₛcₛCsAsT | 66 | 62 |
| SEQ ID NO: 30 | AsCsCstₛcₛcₛtₛgₛaₛcₛaₛcₛtₛTsGsT | 34 | 68 |
| SEQ ID NO: 31 | CsCsAscₛcₛtₛtₛgₛcₛcₛaₛtₛgₛTsTsG | 46 | 70 |
| SEQ ID NO: 32 | TsCsCsaₛcₛcₛtₛtₛgₛcₛcₛaₛtₛGsTsT | 30 | 51 |
| SEQ ID NO: 33 | TsGsCstₛtₛgₛaₛtₛgₛtₛtₛgₛtₛAsGsG | −41 | 83 |
| SEQ ID NO: 34 | CsAsCscₛtₛcₛcₛtₛgₛaₛcₛaₛcₛTsTsG | 13 | 77 |
| SEQ ID NO: 35 | TsGsGscₛaₛcₛaₛaₛgₛgₛaₛcₛaₛTsTsT | −70 | 90 |
| SEQ ID NO: 36 | AsCsTsaₛaₛgₛtₛtₛgₛtₛtₛgₛcₛAsCsC | −16 | 91 |
| SEQ ID NO: 37 | AsTsTsaₛaₛaₛgₛaₛgₛaₛaₛaₛcₛCsTsC | 23 | 81 |
| SEQ ID NO: 38 | TsTsTscₛaₛgₛcₛtₛtₛtₛaₛcₛtₛTₛTₛA | 8 | 90 |
| SEQ ID NO: 39 | CsAsAstₛtₛtₛcₛaₛgₛcₛtₛtₛtₛAsCsT | 7 | 85 |

The data in Table 3 are presented as percentage down-regulation
relative to mock transfected cells at 25 nM. Lower case letters
represent DNA units, bold upper case letters represent p-D-oxy-LNA
units. All LNA C are 5′methyl C. Subscript “s” represents
phosphorothioate linkage.

As shown in Table 3, oligonucleotides of SEQ ID NOs: 33, 34, 35, 36, 37, 38 & 39) demonstrated about 80% or greater inhibition of HSPA2 expression at 25 nM in A549 cells in these experiments and are therefore preferred. As also shown in Table 3, oligonucleotides of SEQ ID NOs: 29, 30, 31 & 32 demonstrated about 30% or greater inhibition of HSPA1A/1B expression at 25 nM in A549 cells in these experiments and are therefore preferred.

Also preferred are oligonucleotides based on the illustrated antisense oligo sequences, for example varying the length (shorter or longer) and/or nucleobase content (e.g. the type and/or proportion of analogue units), which also provide good inhibition of HSPA2 and/or HSPA1A/1B expression.

1-17. (canceled)

18. An oligomer of between 10-30 nucleotides in length comprising a contiguous nucleotide sequence of between 10-30 nucleotides, wherein the contiguous nucleotide sequence is at least 80% homologous to a region corresponding to a mammalian Hsp70-1 RNA or a Hsp70-2 RNA or naturally occurring variant thereof, wherein the contiguous nucleotide sequence comprises at least one Locked Nucleic Acid (LNA) unit.

19. The oligomer according to claim 18, wherein the contiguous nucleotide sequence is at least 80% homologous to a nucleotide sequence selected from the group consisting of SEQ ID NOs 1-3.

20. The oligomer according to claim 19, wherein the contiguous nucleotide sequence comprises at least 10 contiguous nucleotides of SEQ ID NOs 7, 8, 9 10, 18, 19, 20, 21 or 22.

21. The oligomer according to claim 19, wherein the contiguous nucleotide sequence consists of at least 10 contiguous nucleotides of SEQ ID NOs 7, 8, 9 10, 18, 19, 20, 21 or 22.

22. The oligomer according to claim 18, wherein the contiguous nucleotide sequence comprises no mismatches, or no more than one mismatch with the corresponding region of SEQ ID NO 2 or 3.

23. The oligomer according to claim 18, wherein the contiguous nucleotide sequence is at least 80% homologous to a region corresponding to a sequence selected from the group consisting of SEQ ID NO 7-28.

24. The oligomer according to claim 18, wherein the contiguous nucleotide sequence comprises no mismatches, or no more than one mismatch with the corresponding region of SEQ ID NO 1.

25. The oligomer according to claim 18, wherein the nucleotide sequence of the oligomer consists of the contiguous nucleotide sequence.

26. The oligomer according claim 18, wherein the contiguous nucleotide sequence is between 10-18 nucleotides in length.

27. The oligomer according to claim 18, wherein the oligomer is a gapmer.

28. The oligomer according to claim 18, wherein the oligomer inhibits the expression of Hsp70-2 gene or mRNA in a cell which is expressing Hsp70-2 mRNA.

29. A conjugate comprising the oligomer according to claim 18 and at least one non-nucleotide or non-polynucleotide moiety covalently attached to the oligomer.

30. A pharmaceutical composition comprising and oligomer according to claim 18 or a conjugate according to claim 29, and a pharmaceutically acceptable diluent, carrier, salt or adjuvant.

31. A method for treating a hyperproliferative disease, the method comprising administering the oligomer according to claim 18, or the conjugate according to claim 29 to a patient in need thereof

32. A method for the inhibition of Hsp70-2 in a cell which is expressing Hsp70-2, the method comprising administering an oligomer according claim 1 or a conjugate according to claim 29 to the cell so as to inhibit Hsp70-2 in the cell.

33. A method for the simultaneous inhibition of Hsp70-2 and Hsp70-1 in a cell which is expressing both Hsp70-2 and Hsp70-1, the method comprising administering an oligomer according claim 1 or a conjugate according to claim 29 to the cell so as to effect the inhibition of Hsp70-2 and Hsp70-1 in the cell.
